(19) United States
(12) Patent Application Publication
(10) Pub. No.: US 2007/0009466 A1
(43) Pub. Date: Jan. 11, 2007

(54) Durable wrinkle hiding & moisturizing facial cosmetic

(75) Inventors: Francis Busch JR., Southbury, CT (US); Karen A. Bollert, Trumbull, CT (US)

Correspondence Address: THEODORE JAY, SUITE 600, 16 N CHATSWORTH AVE., LARCHMONT, NY, 10538, US

(73) Assignee: PROSTRONG, INC.

(21) Appl. No.: 11/175,462

(22) Filed: Jul. 6, 2005

(51) Int. Cl. A61K 8/89; A61K 8/81; A61K 8/73

(52) U.S. Cl. 424/70.12; 424/70.15; 424/70.17

(57) ABSTRACT

A facial cosmetic containing silica, boron nitride, and a water resistant resin

### FIELD OF THE INVENTION

This invention is directed toward facial cosmetics for diminishing appearance of fine lines and wrinkles.

### BACKGROUND OF THE INVENTION

In view of the importance of improving facial appearance by diminishing the presence of fine lines and wrinkles, a vast variety of cosmetic creams have been developed for this purpose. It is known that crystalline silica used as wrinkle hiding cream is effective, but it has a gray color or cast which must be overcome with a variety of pigments. In addition, the duration of hidden wrinkles when this silica is used is relatively short and moreover to avoid drying of the skin, a separate moisturizing step may be required.

### SUMMARY OF THE INVENTION

In the present invention, the duration of the treatment is substantially increased and at the same time the skin is moisturized and does not dry out. Moreover, the treatment is colorless and uses no pigments. The treatment employs a cosmetic product which is pleasing to the touch and rests comfortably on the skin without flaking. The product can be in the form of a cream, gel or lotion.

The first step in producing this invention is to produce a powder product by blending silica and boron nitride together in proportions by weight of silica 5.0%-15% and by weight of boron nitride 0.7%-2.0%. This powder product is then incorporated into a cosmetic vehicle, as for example a gel, lotion or cream. The cosmetic vehicle is then applied to the skin with the advantages described above.

### DETAILED DESCRIPTION OF PREFERRED EMBODIMENTS

The preferred source of the silica is Malvern Mineral Inc., Novocite 1250, which is well blended with the Boron Nitride in a ribbon blender until uniform. The preferred Boron Nitride is Soft Touch, Grade 6004 from GE Advanced Ceramics. Once the powder phase above is ready, it can be incorporated into a cosmetically acceptable vehicle, for example, a gel, lotion or cream. When a cream is used, it would be made in accordance with the principles of cosmetic emulsion technology well known in the art. These principles are well explained in the standard reference books. A good example is chapter thirty-eight from Harrys' 0 Cosmeticology, Seventh Edition, Chemical Publishing Co. Inc., New York, N.Y. 10011, titled ‘Emulsions’.

When a cream is used, the powder phase is incorporated to the emulsion while hot and with homogenizing. The water-resistant resin, preferably, GE Silicone SF 1318, is incorporated into the oil phase.

The following examples of a cream with silica, boron nitride and water-resistant resin cream are shown in parts per weight: There are four examples each being a different vertical column numbered 1 through 4 respectively.

| | 1 | 2 | 3 | 4 |
|---|---|---|---|---|
| Water Phase | | | | |
| De-ionized water | 61.01 | 65.51 | 52.01 | 64.31 |
| Methylparaben | .1 | .1 | .1 | .1 |
| Propylene Glycol | 5.00 | 5.0 | 5.0 | 5.0 |
| Glycerin | 3.00 | 3.0 | 3.0 | 3.0 |
| Imidazolidinyl Urea | .50 | .5 | .5 | .5 |
| Magnesium Aluminum Silicate | 1.2 | 1.2 | 1.2 | 1.2 |
| Triethanolamine | 0.8 | 0.8 | 0.8 | 0.8 |
| Oil Phase | | | | |
| Cetearyl Alcohol and Cetearth 20 | 1.5 | 1.5 | 1.5 | 1.5 |
| Cetearyl Alcohol | 1.5 | 1.5 | 1.5 | 1.5 |
| Glyceryl Stearate | 1.5 | 1.5 | 1.5 | 1.5 |
| Triceteareth-4 Phosphate | .42 | .42 | .42 | .42 |
| Propylparaben | .20 | .20 | .20 | .20 |
| Myristyl Octanoate | 4.27 | 4.27 | 4.27 | 4.27 |
| Diisostrearyl Trimethylol- | 2.0 | 3.0 | 5.0 | 10.0 |
| Propane Siloxy Silicate | | | | |
| Powder Phase | | | | |
| Silica | 15.0 | 10.0 | 20.0 | 5.0 |
| Boron Nitride | 2.0 | 1.5 | 3.0 | 0.7 |

The Cetearyl alcohol and Cetearth 20 is a commercial blend of ingredients sold by The Dow Corporation under the trade name Promulgen D. The other ingredients are standard cosmetic ingredients widely used and well known in the art and are used for their normal purpose in forming an acceptable cosmetic cream.

Test Results

An independent clinical test of twenty five female subjects (age 35+) with dry skin and lines and wrinkles on the face, yielded the following results.

Skin Moisturization via Impedance Measurement

Shortly (10 minutes) after test cream application, a profound increase (±38.2%) in skin moisturization was found. The magnitude of increases in skin moisturization diminished with time but lasted for at least 8 hours after treatment. At 8 hours post-treatment, an increase of 19.3% in skin moisturization was still evident.

Reduction of Lines and Wrinkles via SFL Scoring, (Packman Method)

Skin Super Facial Lines (SFL) were assessed via the procedure of Packman. The depth, shallowness and the number of SFL's were carefully scored at each time point and total scores were calculated according to the referenced procedure. Clinically, after skin equilibration, average SFL's score of 24.2 was obtained for the 25 subjects. At 10 minutes after a single application of test cream; a significant reduction (−48.3%) in SFLs was observed. At eight hours post-treatment, a 36.5% reduction in SFLs was still evident; suggesting a good duration of effect for the test cream.

While the invention has been described with particular reference to the detailed description, the protection solicited is to be limited only by the terms of the claims that follow.

What is claimed is:

1. A cosmetic powder product adapted for use in producing an anti-wrinkle cosmetic product to be applied to skin, said product comprising a mixture of boron nitride in parts by weight of 2.0% to 0.7% and silica in parts by weight of 15% to 5.0%.

2. A cosmetic powder product according to claim 1 wherein a water resistant resin is added to the said mixture.

3. A cosmetic product comprising silica is used in the range of 1 to 30% by weight, boron nitride in the range of 1 to 30% by weight and a water-resistant resin in the range of 1-10% by weight.

4. The product of claim 3 containing silica 10-20% by weight, boron 1-5% by weight and resin 1-3% by weight.

5. The product according to claim 2, characterized in that said film forming water-resistant resin is in the resin class of silicone based resin, polyvinyl pyrrolidone based resin, and urethane based resin.

6. The product according to claim 5 wherein excipients are added such as moisturizing agents, soothing agents, moistening agents, emollient agents, refreshing agents, thickening agents, preservative agents, stabilizing agents, fragrancing agents.
